(19) United States
(12) Patent Application Publication
(10) Pub. No.: US 2026/0097121 A1
(43) Pub. Date: Apr. 9, 2026

(54) Biallelic Knockout of CISH

(71) Applicant: EmendoBio Inc., Wimington, DE (US)

(72) Inventor: Rafi EMMANUEL, Ramla (IL)

(21) Appl. No.: 19/113,098

(22) PCT Filed: Sep. 18, 2023
(86) PCT No.: PCT/US2023/074470

§ 371 Date: Mar. 19, 2025

Related U.S. Application Data
(60) Provisional application No. 63/376,272, filed on Sep. 19, 2022.

(51) Int. Cl. A61K 40/34; A61K 40/11; C12N 9/22; C12N 15/11; C12N 15/90

(52) U.S. Cl. CPC A61K 40/34; A61K 40/11; C12N 9/226; C12N 15/11; C12N 15/907; C12N 2310/20

(57) ABSTRACT

Compositions comprising an RNA molecule comprising a guide sequence portion having 17-50 contiguous nucleotides containing nucleotides in the sequence set forth in any one of SEQ ID NOs: 1-6838 and methods and uses thereof.

This application is a Section 371 national stage of PCT International Application No. PCT/US2023/074470, filed Sep. 18, 2023, and claims the benefit of U.S. Provisional Application No. 63/376,272 filed Sep. 19, 2022, the content of which is hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “230918_92041-A-PCT_Sequence_Listing_AWG.xml”, which is 5,917 kilobytes in size, and which was created on Sep. 17, 2023 in the IBM-PC machine format, having an operating system compatibility with MS-Windows, which is contained in the XML file filed Mar. 19, 2025 as part of this application.

### BACKGROUND OF INVENTION

Chimeric antigen receptors (CAR) provide a promising approach for immunotherapy. However, to make such therapies (e.g., CAR-T cell therapies) more accessible, it is highly desirable to develop an allogeneic adoptive transfer strategy in which universal CAR cells derived from cells of healthy donors can be applied to treat multiple patients. In order to implement such a strategy, the immunogenicity and reactivity of CAR-T cells must be optimized to avoid harmful reactions, such as rejection by the host or graft-versus-host-disease.

### SUMMARY OF THE INVENTION

Cytokine Inducible SH2 Containing Protein (CISH) belongs to the cytokine-induced STAT inhibitor (CIS) family, which contains cytokine-inducible negative regulators of cytokine signaling. Disclosed are approaches for knocking out the CISH gene in cells to be utilized for immunotherapy approaches, such as CAR-T therapy. A cell modified to have a CISH knockout improves performance of the cell in allogeneic adoptive transfer therapy. Such cells have improved activity, retention, and/or expansion qualities for use in adoptive cancer immunotherapy.

The present disclosure also provides a method for inactivating alleles of the Cytokine Inducible SH2 Containing Protein (CISH) gene in a cell, the method comprising
introducing to the cell a composition comprising:
   a CRISPR nuclease, or a polynucleotide molecule encoding the CRISPR nuclease; and
   an RNA molecule comprising a guide sequence portion having 17-50 nucleotides, or a polynucleotide molecule encoding the RNA molecule,
wherein a complex of the CRISPR nuclease and the RNA molecule affects a double strand break in the allele of the CISH gene.

In some embodiments, the RNA molecule comprises a guide sequence portion that targets a sequence that is located within any one of Exons 1-3 or Intron 2 of the CISH gene, or a sequence that is located within a genomic range selected from any one of 3:50611599-50611805, 3:50608341-50608624, 3:50607575-50608173, and 3:50608112-50608403. In some embodiments, the guide sequence portion of the RNA molecule comprises 17-50 contiguous nucleotides containing nucleotides in the sequence set forth in any one of SEQ ID NOs: 1-6838.

According to embodiments of the present invention, there is provided an RNA molecule comprising a guide sequence portion having 17-50 contiguous nucleotides containing nucleotides in the sequence set forth in any one of SEQ ID Nos: 1-6838.

According to embodiments of the present invention, there is provided a composition comprising an RNA molecule comprising a guide sequence portion having 17-50 contiguous nucleotides containing nucleotides in the sequence set forth in any one of SEQ ID NOs: 1-6838 and a CRISPR nuclease.

According to embodiments of the present invention, there is provided a method for inactivating a CISH allele in a cell, the method comprising delivering to the cell a composition comprising an RNA molecule comprising a guide sequence portion having 17-50 contiguous nucleotides containing nucleotides in the sequence set forth in any one of SEQ ID NOs: 1-6838 and a CRISPR nuclease. In some embodiments, the cell is a lymphocyte. In some embodiments, the cell is a T cell. In some embodiments, the cell is a T regulatory cell. In some embodiments, the cell is a B cell. In some embodiments, the cell is a natural killer (NK) cell. In some embodiments, the cell is a macrophage. In some embodiments, the cell is a stem cell. In some embodiments, the cell is an iPSC. In some embodiments, the cell is a fibroblast, blood cell, hepatocyte, keratinocyte, or any other cell type capable of being reprogrammed to an induced pluripotent stem cell (iPSC). In some embodiments, the delivering to the cell is performed in vivo, ex vivo, or in vitro. In some embodiments, the method is performed ex vivo and the cell is provided/explanted from an individual patient. In some embodiments, the method further comprises the step of introducing the resulting cell, with the modified/knocked out CISH allele, into an individual patient. In some embodiments, the cell is originated from the individual patient to be treated. In some embodiments, the cell is originated from a donor. In some embodiments, the cell is allogeneic to the individual patient to which it is introduced.

According to embodiments of the present invention, there is provided a method for improving the activity and/or retention and/or expansion of a cell for adoptive cell therapy, the method comprising delivering to a cell of a subject in need of the adoptive cell therapy a composition comprising an RNA molecule comprising a guide sequence portion having 17-50 contiguous nucleotides containing nucleotides in the sequence set forth in any one of SEQ ID NOS: 1-6838 and a CRISPR nuclease. In some embodiments, the method is for increasing persistence and/or engraftment of a cell in a host subject, the method comprising delivering to the cell a composition comprising an RNA molecule comprising a guide sequence portion having 17-50 contiguous nucleotides containing nucleotides in the sequence set forth in any one of SEQ ID NOS: 1-6838 and a CRISPR nuclease; and introducing the cell to the host subject. In some embodiments, the cell is further differentiated prior to introducing the cell to the host subject. In some embodiments, the cell is further engineered to express a chimeric antigen receptor. In some embodiments, the cell is a stem cell, an iPSC, or a progenitor cell and is differentiated to a T cell prior to introducing the cell to the host subject. In some embodiments, the cell is a T cell. In some embodiments, the cell is further engineered to have additional genes inactivated and/or knocked-out in order to improve the use of the cell for adoptive transfer, e.g. knockout of additional genes to avoid graft-versus-host disease (GVHD) following introduction of the cell into a host subject.

According to embodiments of the present invention, there is provided use of a composition comprising an RNA molecule comprising a guide sequence portion having 17-50 contiguous nucleotides containing nucleotides in the sequence set forth in any one of SEQ ID NOs: 1-6838 and a CRISPR nuclease for inactivating a CISH allele in a cell, comprising delivering to the cell the composition comprising an RNA molecule comprising a guide sequence portion having 17-50 contiguous nucleotides containing nucleotides in the sequence set forth in any one of SEQ ID NOS: 1-6838 and a CRISPR nuclease.

According to embodiments of the present invention, there is provided a medicament comprising an RNA molecule comprising a guide sequence portion having 17-50 contiguous nucleotides containing nucleotides in the sequence set forth in any one of SEQ ID NOs: 1-6838 and a CRISPR nuclease for use in inactivating a CISH allele in a cell, wherein the medicament is administered by delivering to the cell the composition comprising an RNA molecule comprising a guide sequence portion having 17-50 contiguous nucleotides containing nucleotides in the sequence set forth in any one of SEQ ID NOs: 1-6838 and a CRISPR nuclease.

According to embodiments of the present invention, there is provided use of a composition comprising an RNA molecule comprising a guide sequence portion having 17-50 contiguous nucleotides containing nucleotides in the sequence set forth in any one of SEQ ID NOs: 1-6838 and a CRISPR nuclease for improving the activity and/or retention and/or expansion of a cell for adoptive cell therapy or increasing persistence of the cell upon engraftment, comprising delivering to a cell of a subject in need of the adoptive cell therapy the composition of comprising an RNA molecule comprising a guide sequence portion having 17-50 contiguous nucleotides containing nucleotides in the sequence set forth in any one of SEQ ID NOs: 1-6838 and a CRISPR nuclease.

According to embodiments of the present invention, there is provided a method of treating a disease or disorder, the method comprising delivering any one of the compositions or the modified cells described herein to the subject, preferably wherein the disease or disorder is cancer.

According to embodiments of the present invention, there is provided a kit for inactivating a CISH allele in a cell, comprising an RNA molecule comprising a guide sequence portion having 17-50 contiguous nucleotides containing nucleotides in the sequence set forth in any one of SEQ ID NOs: 1-6838, a CRISPR nuclease, and optionally a tracrRNA molecule; and instructions for delivering the RNA molecule; CRISPR nuclease, and optionally the tracrRNA to the cell.

### BRIEF DESCRIPTION OF THE DRAWINGS

FIG. 1: CISH editing in HeLa cells. OMNI-103 CRISPR nuclease was expressed in a mammalian cell system (HeLa cells) by DNA transfection, together with an sgRNA-expressing plasmid. Transfection efficiency (% transfection) was determined by flow cytometry measurement of mCherry signal. All tests were done in triplicate. ‘OMNI nuclease only’ (i.e. no guide) transfected cells served as a negative control, and no editing was observed in those cells (data not shown).

FIGS. 2A-2B: CISH editing in primary T cells. T cells obtained a donor were thawed and activated with beads for 72 h. Then, a cleavage activity assay was performed with OMNI-103 CRISPR nuclease (113 pmol)+sgRNA (226 pmol) and 2×10⁶ cells per treatment. After seven (7) days, genomic DNA was isolated from ˜100,000 cells and RNA was isolated from ˜1,000,000 cells. Robotic PCR on DNA lysate from Quick Extract was used to prepare next-generation sequencing (NGS) samples for analysis. (FIG. 2A). CISH RNA expression was examined by qPCR (FIG. 2B).

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

In some embodiments of the present invention, a DNA nuclease is utilized to affect a DNA break at a target site to induce cellular repair mechanisms, for example, but not limited to, non-homologous end-joining (NHEJ). During classical NHEJ, two ends of a double-strand break (DSB) site are ligated together in a fast but also inaccurate manner (i.e. frequently resulting in mutation of the DNA at the cleavage site in the form of small insertion or deletions).

As used herein, the term “modified cells” refers to cells in which a double strand break is affected by a complex of an RNA molecule and the CRISPR nuclease as a result of hybridization with the target sequence, i.e. on-target hybridization.

This invention provides a modified cell or cells obtained by use of any of the methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cells (HSCs), or any cell suitable for an allogenic cell transplant or autologous cell transplant.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR nuclease, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-50, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs: 1-6838. Each possibility represents a separate embodiment. In some of these embodiments, the guide sequence portion comprises a sequence that is the same as a sequence set forth in any of SEQ ID NOs: 1-6838. Throughout this application, the terms “guide molecule,” “RNA guide molecule,” “guide RNA molecule,” and “gRNA molecule” are synonymous with a molecule comprising a guide sequence portion.

The term “non-discriminatory” as used herein refers to a guide sequence portion of an RNA molecule that targets a specific DNA sequence that is common to all alleles of a gene.

In embodiments of the present invention, an RNA molecule comprises a guide sequence portion having 17-50 contiguous nucleotides containing nucleotides in the sequence set forth in any one of SEQ ID NOs: 1-6838.

The RNA molecule and/or the guide sequence portion of the RNA molecule may contain modified nucleotides. Exemplary modifications to nucleotides/polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), each of which is hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 17-50 nucleotides in length and contain 20-22 contiguous nucleotides in the sequence set forth in any one of SEQ ID NOs: 1-6838. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-6838. For example, a guide sequence portion having 17 nucleotides in the sequence of 17 contiguous nucleotides set forth in SEQ ID NO: 6834 may consist of any one of the following nucleotide sequences (nucleotides excluded from the contiguous sequence are marked in strike-through):

(SEQ ID NO: 6834)
AAAAAAAUGUACUUGGUUCC
17 nucleotide guide sequence 1:
(SEQ ID NO: 6835) [symbol]
AAAAUGUACUUGGUUCC
17 nucleotide guide sequence 2:
(SEQ ID NO: 6836) [symbol]
AAAAAUGUACUUGGUUC [symbol]
17 nucleotide guide sequence 3:
(SEQ ID NO: 6837) [symbol]
AAAAAAUGUACUUGGUU [symbol]
17 nucleotide guide sequence 4:
(SEQ ID NO: 6838)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24, 25, 26, 27, 28, 29, or 30 nucleotides in length. In such embodiments the guide sequence portion comprises 17-50 nucleotides containing the sequence of 20, 21 or 22 contiguous nucleotides set forth in any one of SEQ ID NOs: 1-6838 and additional nucleotides fully complimentary to a nucleotide or sequence of nucleotides adjacent to the 3′ end of the target sequence, 5′ end of the target sequence, or both.

In embodiments of the present invention, a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRIPSR nuclease binding, e.g. a tracrRNA sequence portion, can be located on the same RNA molecule. Alternatively, a guide sequence portion may be located on one RNA molecule and a sequence portion that participates in CRIPSR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule (e.g. a crRNA molecule) comprising a DNA-targeting RNA portion which includes a guide sequence portion and a separate RNA molecule (e.g. a tracrRNA molecule) comprising a CRISPR protein-binding RNA sequence interact by base pairing to form an RNA complex (e.g. a crRNA:tracrRNA complex) that targets the CRISPR nuclease to a DNA target site or, alternatively, are fused together to form an RNA molecule (e.g. a sgRNA molecule) that complexes with the CRISPR nuclease and targets the CRISPR nuclease to a DNA target site.

In embodiments of the present invention, an RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single guide RNA (sgRNA) molecule. Embodiments of the present invention may also form CRISPR complexes utilizing a separate tracrRNA molecule and a separate RNA molecule comprising a guide sequence portion (e.g. a crRNA molecule). In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

According to embodiments of the present invention, there is provided a method for inactivating alleles of the Cytokine Inducible SH2 Containing Protein (CISH) gene in a cell, the method comprising
introducing to the cell a composition comprising:
   at least one CRISPR nuclease, or a polynucleotide molecule encoding a CRISPR nuclease; and
   an RNA molecule comprising a guide sequence portion, or a polynucleotide molecule encoding the same,
wherein a complex of the CRISPR nuclease and the RNA molecule affects a double strand break in alleles of the CISH gene, and
wherein the guide sequence portion of the RNA molecule comprises 17-50 contiguous nucleotides containing nucleotides in the sequence set forth in any one of SEQ ID NOs: 1-6838.

In some embodiments, the guide sequence portion contains a sequence of nucleotides as listed in SEQ ID NOs: 1-6838, or a portion of any one of SEQ ID NOs: 1-6838, and optionally contains additional nucleotides prepended or appended to the beginning or end of the sequence of nucleotides. As a non-limiting example, a 17-nucleotide sequence found within SEQ ID NO: 1 may form a guide sequence portion. Additionally, a guide sequence portion may comprise a 17-nucleotide sequence found within SEQ ID NO: 1 and further include additional nucleotides 5′ or 3′ of the 17-nucleotide sequence found within SEQ ID NO: 1.

In some embodiments, the RNA molecule is a crRNA molecule and the composition further comprises a tracrRNA molecule that forms a crRNA:tracrRNA complex with the crRNA molecule. In some embodiments, the RNA molecule is an sgRNA molecule.

In some embodiments, the composition comprises additional RNA molecules comprising a guide sequence portion comprising 17-50 contiguous nucleotides containing nucleotides in the sequence set forth in any one of SEQ ID NOs: 1-6838.

In some embodiments, the guide sequence portion of the RNA molecule comprises 17-50 contiguous nucleotides containing nucleotides in the sequence set forth in any one of SEQ ID NOs: 1-6838 modified to comprise up to five mismatches relative to the target site.

In some embodiments, the method is a method of preparing modified immune cells (e.g., T cells) for use in immunotherapy. In some embodiments, the method is performed in vitro or ex vivo.

In some embodiments, the composition is introduced to a cell in a subject or to a cell in culture.

In some embodiments, the cell is a lymphocyte, a T cell, a T regulatory cell, a B cell, a natural killer (NK) cell, a macrophage, a stem cell, or a fibroblast, blood cell, hepatocyte, keratinocyte, or any other cell type capable of being reprogrammed to an induced pluripotent stem cell (iPSC).

In some embodiments, the cell is a hematopoietic stem cell (HSC), induced pluripotent stem cell (iPS cell), iPSc-derived cell, natural killer cell (NK), iPS-derived NK cell (INK), T cell, innate-like T cell (iT), natural killer T cell (NKT), γδ T cell, iPSc-derived T cell, invariant NKT cells (iNKT), iPSc-derived NKT, monocyte, or macrophage.

In some embodiments, the CRISPR nuclease and the RNA molecule are introduced to the cell at substantially the same time or at different times.

In some embodiments, alleles of the CISH gene in the cell are subjected to an insertion or deletion mutation.

In some embodiments, the insertion or deletion mutation creates an early stop codon.

In some embodiments, the inactivating results in a truncated protein encoded by the mutated allele. For example, the method of inactivating mutates a CISH allele such that the mutated allele encodes a truncated form of a CISH protein.

In some embodiments, the composition introduced to the cell further comprises a second RNA molecule comprising a guide sequence portion, wherein the guide sequence portion of the second RNA molecule comprises 17-50 contiguous nucleotides containing nucleotides in the sequence set forth in any one of SEQ ID NOs: 1-6838, preferably wherein the guide sequence portion of the second RNA molecule differs from the guide sequence portion of the first RNA molecule.

According to embodiments of the present invention, there is provided a method for inactivating alleles of the Cytokine Inducible SH2 Containing Protein (CISH) gene in a cell, the method comprising
introducing to the cell a composition comprising:
   at least one CRISPR nuclease, or a polynucleotide molecule encoding a CRISPR nuclease; and
   an RNA molecule comprising a guide sequence portion, or a polynucleotide molecule encoding the RNA molecule,
wherein a complex of the CRISPR nuclease and the RNA molecule affects a double strand break in alleles of the CISH gene,
wherein the guide sequence portion of the RNA molecule comprises 17-50 contiguous nucleotides containing nucleotides in the sequence set forth in any one of SEQ ID NOs: 1-6838 modified to contain 1, 2, 3, 4, or 5 nucleotide mismatches relative to a fully-complementary target sequence of the guide sequence portion.

In some embodiments, the guide sequence portion of the RNA molecule comprises 1, 2, 3, 4, or 5 nucleotide mismatches relative to a fully-complementary target sequence of the guide sequence portion.

In some embodiments, the guide sequence portion provides higher targeting specificity to the complex of the CRISPR nuclease and the RNA molecule relative to a guide sequence portion that has higher complementarity to an allele of the CISH gene.

In some embodiments, the composition introduced to the cell further comprises a second RNA molecule comprising a guide sequence portion, wherein the guide sequence portion of the second RNA molecule comprises 17-50 contiguous nucleotides containing nucleotides in the sequence set forth in any one of SEQ ID NOs: 1-6838, or any one of SEQ ID NOs: 1-6838 modified to contain 1, 2, 3, 4, or 5 nucleotide mismatches relative to a fully-complementary target sequence of the guide sequence portion, preferably wherein the guide sequence portion of the second RNA molecule differs from the guide sequence portion of the first RNA molecule.

According to embodiments of the present invention, there is provided a composition comprising an RNA molecule which comprises a guide sequence portion comprising 17-50 contiguous nucleotides containing nucleotides in the sequence set forth in any one of SEQ ID NOS: 1-6838.

In some embodiments, the composition further comprises a second RNA molecule which comprises a guide sequence portion comprising 17-50 contiguous nucleotides containing nucleotides in the sequence set forth in any one of SEQ ID NOs: 1-6838, preferably wherein the guide sequence portion of the second RNA molecule differs from the guide sequence portion of the first RNA molecule.

According to embodiments of the present invention, there is provided a composition comprising an RNA molecule which comprises a guide sequence portion comprising 17-50 contiguous nucleotides containing nucleotides in the sequence set forth in any one of SEQ ID NOS: 1-6838, or any one of SEQ ID NOs: 1-6838 modified to contain 1, 2, 3, 4, or 5 nucleotide mismatches relative to a fully-complementary target sequence of the guide sequence portion.

In some embodiments, the composition further comprises a second RNA molecule which comprises a guide sequence portion comprising 17-50 contiguous nucleotides containing nucleotides in the sequence set forth in any one of SEQ ID NOs: 1-6838, or any one of SEQ ID NOs: 1-6838 modified to contain 1, 2, 3, 4, or 5 nucleotide mismatches relative to a fully-complementary target sequence of the guide sequence portion, preferably wherein the guide sequence portion of the second RNA molecule differs from the guide sequence portion of the first RNA molecule.

In some embodiments, any one of the compositions described herein further comprises a CRISPR nuclease.

In some embodiments, any one of the compositions described herein further comprises a tracrRNA molecule.

According to embodiments of the present invention, there is provided a cell modified by any one of the methods described herein or modified using the any one of the compositions described herein. The modified cell may also have additional genes altered in order to improve their use for adoptive transfer, for example, reducing or preventing graft-versus-host disease (GVHD).

Preferably all alleles of the CISH gene are inactivated such that the modified cell cannot express a full-length, functional CISH protein product.

In some embodiments, the cell is any one of a wherein the cell is a lymphocyte, a T cell, a T regulatory cell, a B cell, a natural killer (NK) cell, a macrophage, a stem cell, or a fibroblast, blood cell, hepatocyte, keratinocyte, or any other cell type capable of being reprogrammed to an induced pluripotent stem cell (iPSC).

In some embodiments, the cell is a hematopoietic stem cell (HSC), induced pluripotent stem cell (iPS cell), iPSc-derived cell, natural killer cell (NK), iPS-derived NK cell (INK), T cell, innate-like T cell (iT), natural killer T cell (NKT), γδ T cell, iPSc-derived T cell, invariant NKT cell (INKT), iPSc-derived NKT, monocyte, or macrophage.

In some embodiments, the cell is a stem cell or any cell type capable of being reprogrammed to an induced pluripotent stem cell (iPSC).

In some embodiments, the stem cell is differentiated after it is modified.

In some embodiments, the stem cell is differentiated into any one of a lymphocyte, a T cell, a T regulatory cell, a B cell, a natural killer (NK) cell, innate-like T cell (iT), natural killer T cells (NKT), γδ T cell, invariant NKT cells (INKT), monocyte, or macrophage.

According to embodiments of the present invention, there is provided a medicament comprising any one of the compositions described herein for use in inactivating a CISH allele in a cell, wherein the medicament is administered by delivering to the cell the composition.

According to embodiments of the present invention, there is provided a use of any one of the compositions or modified cells described herein for adoptive immunotherapy, for example, to treat cancer.

According to embodiments of the present invention, there is provided a medicament comprising any one of the compositions or modified cells described herein for use in adoptive immunotherapy, for example, to treat cancer.

According to embodiments of the present invention, there is provided a kit for inactivating a CISH allele in a cell, comprising any one of the compositions described herein and instructions for delivering the composition to the cell.

In some embodiments, the composition is delivered to the cell ex vivo.

According to embodiments of the present invention, there is provided a kit for administering adoptive immunotherapy to a subject, comprising any one of the compositions or modified cells described herein and instructions for delivering any one of the compositions or modified cells to a subject in need of adoptive immunotherapy.

According to embodiments of the present invention, there is provided any one of the compositions or modified cells described herein for use in adoptive immunotherapy, comprising delivering any one of the compositions or modified cells described herein to a subject in need of adoptive immunotherapy.

A method of treating a disease or disorder, the method comprising delivering any one of the compositions or modified cells described herein to the subject, preferably wherein the disease or disorder is cancer.

According to embodiments of the present invention, there is provided a composition, method, process, kit, or use as characterized by one or more elements disclosed herein.

According to embodiments of the present invention, the modified immune cells (e.g., T cells) obtained by the described methods are intended to be used as a medicament for treating a cancer, infection, or immune disease in a subject in need thereof. The administration of the modified immune cell or a population thereof to a subject may be carried out in any convenient manner known in the art, including, but not limited to, aerosol inhalation, injection, ingestion, transfusion, implantation or transplantation. Injection or transfusion may be performed subcutaneously, intradermally, intratumorally, intranodally, intramedullary, intramuscularly, by intravenous or intralymphatic injection, or intraperitoneally. According to embodiments of the present invention, there is provided a method for adoptive cell therapy or prophylaxis comprising administering the modified cells to a subject suffering from or determined to be at risk of suffering from a cancer or an infection.

According to embodiments of the present invention, there is provided use of any one of the compositions or modified cells described herein for adoptive immunotherapy, comprising delivering the composition of any one of the compositions or modified cells described herein to a subject in need of adoptive immunotherapy.

According to embodiments of the present invention, there is provided a medicament comprising any one of the compositions or modified cells described herein for use in adoptive immunotherapy, wherein the medicament is administered by delivering any one of the compositions or modified cells described herein to a subject in need of adoptive immunotherapy.

According to embodiments of the present invention, there is provided an RNA molecule for use in modifying a cell (e.g. lymphocyte, T-cell, CAR-T cell) which may be used for adoptive immunotherapy. The RNA molecule may be delivered to the cell ex vivo, in vitro, or in vivo.

According to embodiments of the present invention, there is provided a kit for inactivating a CISH allele in a cell, comprising any one of the compositions described herein and instructions for delivering the composition to the cell.

According to embodiments of the present invention, there is provided a cell modified by the method described herein or modified using the compositions described herein.

According to embodiments of the present invention, there is provided a gene editing composition comprising an RNA molecule comprising a guide sequence portion having 17-50 contiguous nucleotides containing nucleotides in the sequence set forth in any one of SEQ ID NOS: 1-6838. In some embodiments, the RNA molecule further comprises a portion having a sequence which binds to a CRISPR nuclease. In some embodiments, the sequence which binds to a CRISPR nuclease is a tracrRNA sequence. In some embodiments the RNA comprising a guide sequence portion is a crRNA molecule. In some embodiments an RNA molecule comprising a guide sequence portion is a single-guide RNA (sgRNA) molecule.

In some embodiments, the RNA molecule further comprises a portion having a tracr mate sequence.

In some embodiments, the RNA molecule may further comprise one or more linker portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 100 up to 500 nucleotides in length, 100 up to 400 nucleotides in length, 200 up to 300 nucleotides in length, 100 to 200 nucleotides in length, or 150 up to 250 nucleotides in length. Each possibility represents a separate embodiment.

According to some embodiments of the present invention, the composition further comprises a tracrRNA molecule.

According to some embodiments of the present invention, there is provided a method for inactivating CISH expression in a cell, the method comprising delivering to the cell a composition comprising an RNA molecule comprising a guide sequence portion having 17-50 contiguous nucleotides containing nucleotides in the sequence set forth in any one of SEQ ID NOs: 1-6838 and a CRISPR nuclease.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

The compositions and methods of the present disclosure may be utilized for improved adoptive immunotherapy.

Any one of, or combination of, the strategies for deactivating CISH expression described herein may be used in the context of the invention.

In embodiments of the present invention, a guide RNA molecule is used to direct a CRISPR nuclease to an exon or a splice site of a CISH allele in order to create a double-stranded break (DSB), leading to insertion or deletion of nucleotides by inducing an error-prone non-homologous end-joining (NHEJ) mechanism and formation of a frameshift mutation in the CISH allele. The frameshift mutation may result in, for example, inactivation or knockout of the CISH allele by generation of an early stop codon in the CISH allele and to generation of a truncated protein or to nonsense-mediated mRNA decay of the transcript of the allele. In further embodiments, one RNA molecule is used to direct a CRISPR nuclease to a promotor of a CISH allele.

Embodiments of compositions described herein include at least one CRISPR nuclease, guide RNA molecule(s), and optionally a tracrRNA molecule(s), being effective in a subject or cells at the same time. The at least one CRISPR nuclease, guide RNA molecule(s), and optional tracrRNA molecule(s) may be delivered substantially at the same time or can be delivered at different times but have effect at the same time. For example, this includes delivering the CRISPR nuclease to the subject or cells before the guide RNA molecule and/or tracrRNA molecule is substantially extant in the subject or cells.

In some embodiments, the cell is a lymphocyte. In some embodiments, the cell is a T cell. In some embodiments, the cell is a T regulatory cell. In some embodiments, the cell is a B cell. In some embodiments, the cell is a natural killer (NK) cell. In some embodiments, the cell is a macrophage. In some embodiments, the cell is a stem cell. In some embodiments, the cell is a fibroblast, blood cell, hepatocyte, keratinocyte, or any other cell type capable of being reprogrammed to an induced pluripotent stem cell (iPSC).

### CISH Editing Strategies

The present invention provides methods to knockout CISH alleles in cells of a subject, thereby improving the performance of the cells, or cells derived therefrom, in adoptive transfer therapies.

CISH editing strategies include, but are not limited to, biallelic knockout by targeting any one of, or a combination of, Exons 1-3 and Intron 2, including within thirty nucleotides upstream and downstream of the exons to flank splice donor and acceptor sites, as frameshifts in these exons lead to non-functional, truncated CISH proteins or non-sense mediated decay of the mutated CISH transcripts.

### CRISPR Nucleases and PAM Recognition

In some embodiments, the sequence specific nuclease is selected from CRISPR nucleases, or is a functional variant thereof. In some embodiments, the sequence specific nuclease is an RNA guided DNA nuclease. In such embodiments, the RNA sequence which guides the RNA guided DNA nuclease (e.g., Cpf1) binds to and/or directs the RNA guided DNA nuclease to all CISH alleles in a cell. In some embodiments, the CRISPR complex does not further comprise a tracrRNA. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer.

In a non-limiting example, a type II CRISPR system utilizes a mature crRNA:tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NRRH for SpCas9-NRRH variant, wherein N is any nucleobase, R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase; NG or NA for SpCas9-NG variant of SpCas9 wherein N is any nucleobase; NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpf1, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cashe, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention. Additional CRISPR nucleases may also be used, for example, the nucleases described in PCT International Application Publication Nos. WO2020/223514 and WO2020/223553, which are hereby incorporated by reference.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Derivatives include, but are not limited to, CRISPR nickases, catalytically inactive or “dead” CRISPR nucleases, and fusion of a CRISPR nuclease or derivative thereof to other enzymes such as base editors or retrotransposons. See for example, Anzalone et al. (2019) and PCT International Application No. PCT/US2020/037560.

In some embodiments, the CRISPR nuclease or derivative thereof may be fused to a protein that has an enzymatic activity. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the enzymatic activity is nuclease activity. In some cases, the nuclease activity introduces a double strand break in the target DNA. In some cases, the enzymatic activity modifies a target polypeptide associated with the target DNA. In some cases, the enzymatic activity is methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity. In some cases, the target polypeptide is a histone and the enzymatic activity is methyltransferase activity, demethylase activity, acetyltransferase activity, deacetylase activity, kinase activity, phosphatase activity, ubiquitin ligase activity or deubiquitinating activity.

Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas.

In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl) uridine, 2′-O-methylcytidine. 5-carboxymethylaminomethyl-2-thiouridine, 5-carboxymethylaminomethyluridine, dihydrouridine, 2′-O-methylpseudouridine. “beta, D-galactosylqueuosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid-methylester, uridine-S-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, “3-(3-amino-3-carboxy-propyl)uridine, (acp3) u”, 2′-O-methyl (M), 3′-phosphorothioate (MS), 3′-thioPACE (MSP), pseudouridine, or 1-methyl pseudo-uridine. Each possibility represents a separate embodiment of the present invention.

In addition to targeting CISH alleles by a RNA-guided CRISPR nuclease, other means of inhibiting CISH expression in a target cell include but are not limited to use of a gapmer, shRNA, siRNA, a customized TALEN, meganuclease, or zinc finger nuclease, a small molecule inhibitor, and any other method known in the art for reducing or eliminating expression of a gene in a target cell. See, for example, U.S. Pat. Nos. 6,506,559; 7,560,438; 8,420,391; 8,552,171; 7,056,704; 7,078,196; 8,362,231; 8,372,968; 9,045,754; and PCT International Publication Nos. WO/2004/067736; WO/2006/097853; WO/2003/087341; WO/2000/0415661; WO/2003/080809; WO/2010/079430; WO/2010/079430; WO/2011/072246; WO/2018/057989; and WO/2017/164230, the entire contents of each of which are incorporated herein by reference.

Advantageously, the guide RNA molecules presented herein provide improved CISH knockout efficiency when complexed with a CRISPR nuclease in a cell relative to other guide RNA molecules. These specifically designed sequences may also be useful for identifying CISH target sites for other nucleotide targeting-based gene-editing or gene-silencing methods, for example, siRNA, TALENs, meganucleases or zinc-finger nucleases.

### Delivery to Cells

Any one of the compositions described herein may be delivered to a target cell by any suitable means. RNA molecule compositions of the present invention may be targeted to any cell which contains and/or expresses a CISH allele, such as a mammalian lymphocyte or stem cell. For example, in one embodiment the RNA molecule specifically targets CISH alleles in a target cell and the target cell is a lymphocyte, a T cell, a T regulatory cell, a B cell, a natural killer (NK) cell, a macrophage, a stem cell, or a fibroblast, blood cell, hepatocyte, keratinocyte, or any other cell type capable of being reprogrammed to an induced pluripotent stem cell (iPSC). The delivery to the cell may be performed in vivo, ex vivo, or in vitro. Further, the nucleic acid compositions described herein may be delivered to a cell as one or more of DNA molecules, RNA molecules, ribonucleoproteins (RNP), nucleic acid vectors, or any combination thereof.

In some embodiments, the RNA molecule comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention.

In some embodiments, any one of the compositions described herein is delivered to a cell in-vivo. The composition may be delivered to the cell by any known in-vivo delivery method, including but not limited to, viral transduction, for example, using a lentivirus or adeno-associated virus (AAV), nanoparticle delivery, etc. Additional detailed delivery methods are described throughout this section.

In some embodiments, any one of the compositions described herein is delivered to a cell ex-vivo. The composition may be delivered to the cell by any known ex-vivo delivery method, including but not limited to, nucleofection, electroporation, viral transduction, for example, using a lentivirus or adeno-associated virus (AAV), nanoparticle delivery, liposomes, etc. Additional detailed delivery methods are described throughout this section.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993); Miller (1992); Van Brunt (1988); Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboium meliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam™, Lipofectin™, and Lipofectamine™ RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

Delivery vehicles include, but are not limited to, bacteria, preferably non-pathogenic, vehicles, nanoparticles, exosomes, microvesicles, gene gun delivery, for example, by attachment of a composition to a gold particle which is fired into a cell using via a “gene-gun”, viral vehicles, including but not limited to lentiviruses, AAV, and retroviruses), virus-like particles (VLPs). large VLPs (LVLPs), lentivirus-like particles, transposons, viral vectors, naked vectors, DNA, or RNA, among other delivery vehicles known in the art.

The delivery of a CRISPR nuclease and/or a polynucleotide encoding the CRIPSR nuclease, and optionally additional nucleotide molecules and/or additional proteins or peptides, may be performed by utilizing a single delivery vehicle or method or a combination of different delivery vehicles or methods. For example, a CRISPR nuclease may be delivered to a cell utilizing an LNP, and a crRNA molecule and tracrRNA molecule may be delivered to the cell utilizing AAV. Alternatively, a CRISPR nuclease may be delivered to a cell utilizing an AAV particle, and a crRNA molecule and tracrRNA molecule may be delivered to the cell utilizing a separate AAV particle, which may be advantageous due to size limitations.

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer. An RNA virus may be utilized for delivery of the compositions described herein. Additionally, high transduction efficiencies have been observed in many different cell types and target tissues. Nucleic acid of the invention may be delivered by non-integrating lentivirus. Optionally, RNA delivery with Lentivirus is utilized. Optionally the lentivirus includes mRNA of the nuclease and a guide RNA molecule. Optionally, the lentivirus includes the nuclease protein and a guide RNA molecule. Optionally the lentivirus includes mRNA of the nuclease, a guide RNA molecule, and a tracrRNA molecule. Optionally, the lentivirus includes the nuclease protein, a guide RNA molecule, and a tracrRNA molecule.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that

Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, for example by systemic administration (e.g., intravitreal, intravenous, intraperitoneal, intramuscular, subdermal, or intracranial infusion) or topical application.

Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Patent Publication No. 2009/0117617.

As mentioned above, the compositions described herein may be delivered to a target cell using a non-integrating lentiviral particle method, e.g. a LentiFlash® system. Such a method may be used to deliver mRNA or other types of RNAs into the target cell, such that delivery of the RNAs to the target cell results in assembly of the compositions described herein inside of the target cell. See also PCT International Publication Nos. WO/2013/014537, WO/2014/016690, WO/2016/185125, WO/2017/194902, and WO/2017/194903.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

Examples of RNA Guide Sequences which Specifically Target Alleles of CISH Gene

Although a large number of guide sequences can be designed to target the CISH gene, the nucleotide sequences described in Table 2 and are identified by SEQ ID NOs: 1-6833 were specifically selected to effectively implement the methods set forth herein.

Table 2 lists guide sequences designed for use as described in the embodiments above to associate specific sequences within a CISH allele. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpf1 (PAM SEQ: TTTV), JeCas9WT (PAM SEQ: NNNVRYM), OMNI-50 (PAM SEQ: NGG), OMNI-79 (PAM SEQ: NGG), OMNI-103 (PAM SEQ: NNRACT), OMNI-159 (NNNNCMAN), or OMNI-124 (PAM SEQ: NNGNRMNN).

Additional description of OMNI CRISPR nucleases is provided in PCT International Application Publication No. WO 2023/019269 A2, PCT International Application Publication Nos. WO 2022/170199 A2 and WO 2023/107946 A2, U.S. Pat. No. 11,666,641 B2 and PCT International Application Publication Nos. WO 2020/223514 A2, WO 2022/098693 A1, and WO 2023/019263 A1, U.S. Application Publication No. 2023/0122086 A1 and PCT International Application Publication Nos. WO 2021/248016 A2 and WO 2023/102407 A2, PCT International Application Publication No. WO 2022/087135 A1, and PCT International Application Publication No. WO 2022/226215 A1, the contents of each of which are hereby incorporated by reference.

RNA molecules of the present invention are each designed to form complexes in conjunction with one or more different CRISPR nucleases and designed to target polynucleotide sequences of interest utilizing one or more different PAM sequences respective to the CRISPR nuclease utilized.

As used herein, the following nucleotide identifiers are used to represent a referenced nucleotide base(s):

TABLE 1

| | |
|---|---|
| Nucleotide | |
| reference | Base(s) represented |

| | | | | |
|---|---|---|---|---|
| A | A | | | |
| C | | C | | |
| G | | | G | |
| T | | | | T |
| W | A | | | T |
| S | | C | G | |
| M | A | C | | |
| K | | | G | T |
| R | A | | G | |
| Y | | C | | T |
| B | | C | G | T |
| D | A | | G | T |
| H | A | C | | T |
| V | A | C | G | |
| N | A | C | G | T |

TABLE 2
Guide sequence portions designed to associate with specific CISH gene targets

| | SEQ ID NOs: of | SEQ ID NOs: of | SEQ ID NOs: of |
|---|---|---|---|
| Target | 20 base guides | 21 base guides | 22 base guides |
| 3:50611599-50611805 | 1-302 | 303-550 | 551-820 |
| Exon 1, including 30 nt | | | |
| upstream and 30 nt | | | |
| downstream of the exon | | | |
| 3:50608341-50608624 | 821-1314 | 1315-1790 | 1791-2270 |
| Exon 2, including 30 nt | | | |
| upstream and 30 nt | | | |
| downstream of the exon | | | |
| 3:50607575-50608173 | 2271-3410 | 3411-4530 | 4531-5660 |
| Exon 3, including 30 nt | | | |
| upstream and 30 nt | | | |
| downstream of the exon | | | |
| 3:50608112-50608403 | 821, 823, 826, | 1315, 1317, 1320, | 1791, 1793, |
| Intron 2 | 830, 835, 840, | 1324, 1329, 1334, | 1796, 1800, |
| | 846-847, 850, | 1340-1341, 1344, | 1805, 1810, |
| | 862-863, 869, | 1356-1357, 1363, | 1816-1817, |
| | 881, 890, 902, | 1374, 1383, 1393, | 1820, 1831- |
| | 909, 913, 916, | 1400, 1407, 1413, | 1832, 1838, |
| | 922, 930, 935, | 1421, 1426, 1443, | 1850, 1859, |
| | 954, 963, 980- | 1452, 1467-1468, | 1876, 1883, |
| | 981, 983, 987, | 1470, 1473, 1481- | 1889, 1897, |
| | 996-998, 1006, | 1483, 1491, 1496, | 1902, 1919, |
| | 1011, 1013, | 1498, 1508, 1511, | 1928, 1944- |
| | 1024, 1027, | 1515, 1518, 1522- | 1945, 1947, |
| | 1031, 1034, | 1523, 1537, 1550, | 1950, 1959- |
| | 1038-1039, | 1556, 1558, 1564, | 1961, 1969, |
| | 1053, 1067, | 1569, 1573, 1575, | 1974, 1976, |
| | 1073, 1075, | 1588, 1592, 1605, | 1987, 1990, |
| | 1081, 1087, | 1616-1617, 1634, | 1994, 1997, |
| | 1091, 1093, | 1637, 1650, 1658, | 2001-2002, |
| | 1107, 1111, | 1675, 1690, 1699, | 2016, 2029, |
| | 1124, 1135- | 1705, 1708, 1716, | 2035, 2037, |
| | 1136, 1153, | 1720, 1728, 1732, | 2043, 2048, |
| | 1156, 1171, | 1738, 1743, 1751, | 2052, 2054, |
| | 1179, 1197, | 1773, 1781, 1783, | 2067, 2084, |
| | 1209, 1213, | 3421, 3439, 3441, | 2095-2096, |
| | 1222, 1228, | 3467, 3470, 3515, | 2113, 2116, |
| | 1231, 1239, | 3537, 3555, 3568, | 2130, 2138, |
| | 1243, 1251, | 3597, 3618, 3621, | 2155, 2170, |
| | 1255, 1261, | 3637, 3668, 3684, | 2179, 2185, |
| | 1266, 1274, | 3727, 3745, 3755, | 2188, 2196, |
| | 1297, 1305, | 3760, 3763, 3768, | 2200, 2208, |
| | 1307, 2281, | 3777, 3780, 3797, | 2212, 2218, |
| | 2299, 2301, | 3803-3804, 3812, | 2223, 2231, |
| | 2327, 2330, | 3826, 3850, 3870, | 2253, 2261, |
| | 2376, 2398, | 3891, 3893, 3909, | 2263, 4541, |
| | 2416, 2430, | 3918, 3936, 3941, | 4559, 4561, |
| | 2459, 2480, | 3961-3962, 3971, | 4587, 4590, |
| | 2483, 2499, | 4029, 4065, 4079, | 4635, 4657, |
| | 2531, 2547, | 4081-4082, 4116, | 4688, 4717, |
| | 2591, 2610, | 4119, 4130, 4166, | 4738, 4741, |
| | 2620, 2625, | 4172, 4183, 4231, | 4757, 4789, |
| | 2628, 2633, | 4258, 4309, 4315, | 4805, 4849, |
| | 2642, 2645, | 4329, 4333, 4364, | 4867, 4877, |
| | 2662, 2668- | 4375, 4386, 4410, | 4882, 4885, |
| | 2669, 2677, | 4425, 4443, 4454, | 4890, 4899, |
| | 2692, 2717, | 4457, 4469, 4485, | 4902, 4919, |
| | 2738, 2759, | 4515, 4517, and | 4925-4926, |
| | 2761, 2777, | 6049-6438 | 4934, 4948, |
| | 2786, 2807, | | 4973, 4993, |
| | 2812, 2832- | | 5014, 5016, |
| | 2833, 2842, | | 5032, 5041, |
| | 2900, 2939, | | 5060, 5065, |
| | 2953, 2955- | | 5085-5086, |
| | 2956, 2990, | | 5095, 5153, |
| | 2993, 3004, | | 5190, 5204, |
| | 3022, 3042, | | 5206-5207, |
| | 3048, 3059, | | 5243, 5246, |
| | 3090, 3107, | | 5257, 5293, |
| | 3134, 3186, | | 5299, 5310, |
| | 3192, 3206, | | 5358, 5385, |
| | 3210, 3241, | | 5437, 5443, |
| | 3252, 3263, | | 5456, 5460, |
| | 3287, 3302, | | 5491, 5502, |
| | 3323, 3334, | | 5513, 5537, |
| | 3337, 3349, | | 5552, 5573, |
| | 3365, 3395, | | 5584, 5587, |
| | 3397, and | | 5615, 5645, |
| | 5661-6048 | | 5647, and |
| | | | 6439-6830 |

The indicated locations listed in column 1 of the Table 2 are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1: CISH Modification Analysis

Guide sequences comprising 17-50 contiguous nucleotides containing nucleotides in the sequence set forth in any one of SEQ ID NOs: 1-6833 are screened for high on target activity using a CRISPR nuclease in T cells. On target activity is determined by DNA capillary electrophoresis analysis.

### Example 2: CISH Editing in HeLa and Primary T Cells

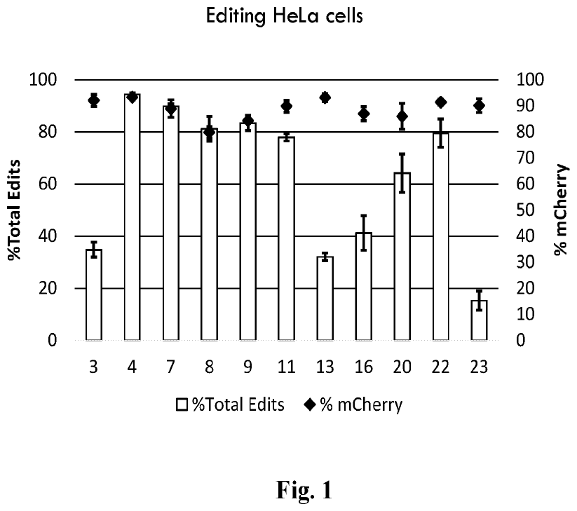

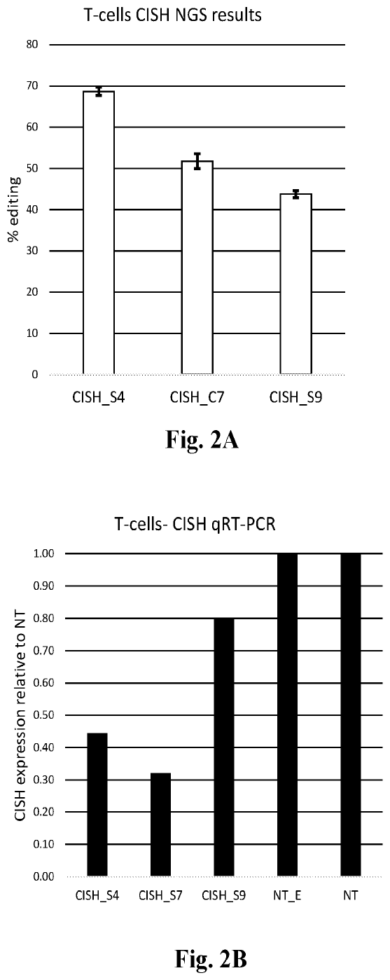

CISH editing in HeLa cells and primary T cells using a subset of the disclosed CISH-targeting guide sequence portions are shown in FIG. 1 and FIG. 2.

A summary table of the CISH editing data is shown below:

TABLE 3

| | | | % editing | % editing |
|---|---|---|---|---|
| Target | | | NGS | NGS |
| Site | Spacer Name | Guide Sequence Portion | (HeLa) | (T cells) |
| 3 | hCISH_S3_22bp_II | CUCGCCCUCGCAGGCGGUCCGC | 34.82 | |
| | | (SEQ ID NO: 6831) | | |
| 4 | hCISH_S4_22bp_II | CGGGGCGCGCGGGCGCAGGACA | 94.39 | 68.65 |
| | | (SEQ ID NO: 6832) | | |
| 7 | hCISH_S7_22bp_II | CGGCAGCGGCGACUCCGGAGUG | 89.76 | 51.71 |
| | | (SEQ ID NO: 658) | | |
| 8 | hCISH_S8_22bp_II | GGACCAUGUCCCCGCGGCAGCG | 81.17 | |
| | | (SEQ ID NO: 746) | | |
| 9 | hCISH_S9_22bp_II | GGGAGGGCUUGACGGGCCAGAG | 83.45 | 43.745 |
| | | (SEQ ID NO: 6833) | | |
| 11 | hCISH_S11_22bp_II | UCGUCCUUUGCUGGCUGUGGAG | 77.91 | |
| | | (SEQ ID NO: 2209) | | |
| 13 | hCISH_S13_22bp_II | GGCCCCAGCAGGCAAGGGCUGC | 32.10 | |
| | | (SEQ ID NO: 2127) | | |
| 16 | hCISH_S16_22bp_II | CACUAUGUCCCUUGGCCCCCUC | 41.19 | |
| | | (SEQ ID NO: 6537) | | |
| 20 | hCISH_S20_22bp_II | ACCAAUGUACGCAUUGAGUAUG | 64.21 | |
| | | (SEQ ID NO: 4582) | | |
| 22 | hCISH_S22_22bp_II | UAUGCCGACUCCAGCUUCCGUC | 79.55 | |
| | | (SEQ ID NO: 5467) | | |

OMNI-103 related sequences are provided in the table below:

TABLE 4

| | | OMNI-103 DNA | |
|---|---|---|---|
| | | sequence codon | OMNI-103 |
| OMNI-103 | OMNI-103 | optimized for | sgRNA |
| Amino Acid | DNA | expression | scaffold |
| Sequence | sequence | in human cells | sequence |
| SEQ ID NO: | SEQ ID NO: | SEQ ID NO: | SEQ ID NO: |
| 6839 | 6840 | 6841 | 6842 |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Anzalone et al. (2019) “Search-and-replace genomme editing without double-strand brakes or donor DNA”, Nature 576, 149-157.
4. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
5. Behr (1994) Gene transfer with synthetic cationic amphiphiles: Prospects for gene therapy”, Bioconjuage Chem 5:382-89.
6. Blaese (1995) “Vectors in cancer therapy: how will they deliver”, Cancer Gene Ther. 2:291-97.
7. Blaese et al. (1995) “T lympocyte-directed gene therapy for ADA-SCID: initial trial results after 4 years”, Science 270(5235):475-80.
8. Buchschacher and Panganiban (1992) “Human immunodeficiency virus vectors for inducible expression of foreign genes”, J. Virol. 66:2731-39.
9. Burstein et al. (2017) “New CRISPR-Cas systems from uncultivated microbes”, Nature 542:237-41.
10. Chang and Wilson (1987) “Modification of DNA ends can decrease end-joining relative to homologous recombination in mammalian cells”, Proc. Natl. Acad. Sci. USA 84:4959-4963.
11. Chung et al. (2006) “Agrobacterium is not alone: gene transfer to plants by viruses and other bacteria”, Trends Plant Sci. 11(1):1-4.
12. Coelho et al. (2013) “Safety and efficacy of RNAi therapy for transthyretin amyloidosis” N. Engl. J. Med. 369, 819-829.
13. Collias and Beisel (2021) “CRISPR technologies and the search for the PAM-free nuclease”, Nature Communications 12, 555.
14. Crystal (1995) “Transfer of genes to humans: early lessons and obstacles to success”, Science 270(5235):404-10.
15. Dillon (1993) “Regulation gene expression in gene therapy” Trends in Biotechnology 11(5):167-173.
16. Dranoff et al. (1997) “A phase I study of vaccination with autologous, irradiated melanoma cells engineered to secrete human granulocyte macrophage colony stimulating factor”, Hum. Gene Ther. 8(1):111-23.
17. Dunbar et al. (1995) “Retrovirally marked CD34-enriched peripheral blood and bone marrow cells contribute to long-term engraftment after autologous transplantation”, Blood 85:3048-57.
18. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
19. Gao and Huang (1995) “Cationic liposome-mediated gene transfer” Gene Ther. 2(10):710-22.
20. Haddada et al. (1995) “Gene Therapy Using Adenovirus Vectors”, in: The Molecular Repertoire of Adenoviruses III: Biology and Pathogenesis, ed. Doerfler and Böhm, pp. 297-306.
21. Han et al. (1995) “Ligand-directed retro-viral targeting of human breast cancer cells”, Proc Natl Acad Sci U.S.A. 92(21):9747-51.
22. Hu et al. (2018) “Evolved Cas9 variants with broad PAM compatibility and high DNA specificity”, Nature 556(7699):57-63.
23. Jinek et al. (2012) “A programmable dual-RNA-guided DNA endonuclease in adaptive bacterial immunity,” Science 337(6096):816-21.
24. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
25. Judge et al. (2006) “Design of noninflammatory synthetic siRNA mediating potent gene silencing in vivo”, Mol Ther. 13(3):494-505.
26. Kohn et al. (1995) “Engraftment of gene-modified umbilical cord blood cells in neonates with adnosine deaminase deficiency”, Nature Medicine 1:1017-23.
27. Kremer and Perricaudet (1995) “Adenovirus and adeno-associated virus mediated gene transfer”, Br. Med. Bull. 51(1):31-44.
28. Macdiarmid et al. (2009) “Sequential treatment of drug-resistant tumors with targeted minicells containing siRNA or a cytotoxic drug”, Nat Biotechnol. 27(7):643-51.
29. Malech et al. (1997) “Prolonged production of NADPH oxidase-corrected granulocytes after gene therapy of chronic granulomatous disease”, PNAS 94(22):12133-38.
30. Miller et al. (1991) “Construction and properties of retrovirus packaging cells based on gibbon ape leukemia virus”, J Virol. 65(5):2220-24.
31. Miller (1992) “Human gene therapy comes of age”, Nature 357:455-60.
32. Mitani and Caskey (1993) “Delivering therapeutic genes-matching approach and application”, Trends in Biotechnology 11(5):162-66.
33. Nabel and Felgner (1993) “Direct gene transfer for immunotherapy and immunization”, Trends in Biotechnology 11(5):211-15.
34. Nishimasu et al. (2018) “Engineered CRISPR-Cas9 nuclease with expanded targeting space”, Science 361(6408):1259-1262.
35. Remy et al. (1994) “Gene Transfer with a Series of Lipphilic DNA-Binding Molecules”, Bioconjugate Chem. 5(6):647-54.
36. Sentmanat et al. (2018) “A Survey of Validation Strategies for CRISPR-Cas9 Editing”, Scientific Reports 8:888, doi:10.1038/s41598-018-19441-8.
37. Sommerfelt et al. (1990) “Localization of the receptor gene for type D simian retroviruses on human chromosome 19”, J. Virol. 64(12):6214-20.
38. Van Brunt (1988) “Molecular framing: transgenic animals as bioactors” Biotechnology 6:1149-54.
39. Vigne et al. (1995) “Third-generation adenovectors for gene therapy”, Restorative Neurology and Neuroscience 8(1,2):35-36.
40. Walton et al. (2020) “Unconstrained genome targeting with near-PAMless engineered CRISPR-Cas9 variants”, Science 368(6488):290-296.
41. Weissman and Kariko (2015) “mRNA: Fulfilling the promise of gene therapy”, Molecular Therapy (9):1416-7.
42. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
43. Yu et al. (1994) “Progress towards gene therapy for HIV infection”, Gene Ther. 1(1):13-26.
44. Zetsche et al. (2015) “Cpf1 is a single RNA-guided endonuclease of a class 2 CRIPSR-Cas system” Cell 163(3):759-71.
45. Zuris et al. (2015) “Cationic lipid-mediated delivery of proteins enables efficient protein based genome editing in vitro and in vivo” Nat Biotechnol. 33(1):73-80.

1. A method for inactivating alleles of the Cytokine Inducible SH2 Containing Protein (CISH) gene in a cell, the method comprising
introducing to the cell a composition comprising:
at least one CRISPR nuclease, or a polynucleotide molecule encoding a CRISPR nuclease; and
an RNA molecule comprising a guide sequence portion, or a polynucleotide molecule encoding the RNA molecule,
wherein a complex of the CRISPR nuclease and the RNA molecule affects a double strand break in alleles of the CISH gene, and
wherein the guide sequence portion of the RNA molecule comprises 17-50 contiguous nucleotides containing nucleotides in the sequence set forth in any one of SEQ ID NOs: 1-6838.

2. (canceled)

3. The method of claim 1, wherein the cell is a lymphocyte, a T cell, a T regulatory cell, a B cell, a natural killer (NK) cell, a macrophage, a stem cell, or a fibroblast, blood cell, hepatocyte, keratinocyte, or any other cell type capable of being reprogrammed to an induced pluripotent stem cell (iPSC), or
wherein the cell is a hematopoietic stem cell (HSC), induced pluripotent stem cell (iPS cell), iPSc-derived cell, natural killer cell (NK), iPS-derived NK cell (INK), T cell, innate-like T cell (iT), natural killer T cell (NKT), γδ T cell, iPSc-derived T cell, invariant NKT cells (INKT), iPSc-derived NKT, monocyte, or macrophage.

4. (canceled)

5. (canceled)

6. The method of claim 1, wherein alleles of the CISH gene in the cell are subjected to an insertion or deletion mutation.

7. (canceled)

8. (canceled)

9. The method of claim 1, wherein the composition introduced to the cell further comprises a second RNA molecule comprising a guide sequence portion, wherein the guide sequence portion of the second RNA molecule comprises 17-50 contiguous nucleotides containing nucleotides in the sequence set forth in any one of SEQ ID NOs: 1-6838, wherein the guide sequence portion of the second RNA molecule differs from the guide sequence portion of the first RNA molecule.

10. A method for inactivating alleles of the Cytokine Inducible SH2 Containing Protein (CISH) gene in a cell, the method comprising
introducing to the cell a composition comprising:
at least one CRISPR nuclease, or a polynucleotide molecule encoding a CRISPR nuclease; and
an RNA molecule comprising a guide sequence portion, or a polynucleotide molecule encoding the RNA molecule,
wherein a complex of the CRISPR nuclease and the RNA molecule affects a double strand break in alleles of the CISH gene, and
wherein the guide sequence portion of the RNA molecule comprises 17-50 contiguous nucleotides containing nucleotides in the sequence set forth in any one of SEQ ID NOs: 1-6838 modified to contain 1, 2, 3, 4, or 5 nucleotide mismatches relative to a fully-complementary target sequence of the guide sequence portion.

11. (canceled)

12. The method of claim 10, wherein the guide sequence portion provides higher targeting specificity to the complex of the CRISPR nuclease and the RNA molecule relative to a guide sequence portion that has higher complementarity to an allele of the CISH gene.

13. The method of claim 10, wherein the composition introduced to the cell further comprises a second RNA molecule comprising a guide sequence portion, wherein the guide sequence portion of the RNA molecule comprises 17-50 contiguous nucleotides containing nucleotides in the sequence set forth in any one of SEQ ID NOs: 1-6838, or any one of SEQ ID NOs: 1-6838 modified to contain 1, 2, 3, 4, or 5 nucleotide mismatches relative to a fully-complementary target sequence of the guide sequence portion, wherein the guide sequence portion of the second RNA molecule differs from the guide sequence portion of the first RNA molecule.

14. A composition comprising:
at least one CRISPR nuclease, or a polynucleotide molecule encoding a CRISPR nuclease; and
an RNA molecule comprising a guide sequence portion, or a polynucleotide molecule encoding the RNA molecule,
wherein the guide sequence portion of the RNA molecule comprises 17-50 contiguous nucleotides containing nucleotides in the sequence set forth in any one of SEQ ID NOs: 1-6838 or in any one of SEQ ID NOs: 1-6838, modified to contain 1, 2, 3, 4, or 5 nucleotide mismatches relative to a fully target sequence of the guide sequence portion.

15. The composition of claim 14, further comprising a second RNA molecule which comprises a guide sequence portion comprising 17-50 contiguous nucleotides containing nucleotides in the sequence set forth in any one of SEQ ID NOs: 1-6838, or any one of SEQ ID NOs: 1-6838 modified to contain 1, 2, 3, 4, or 5 nucleotide mismatches relative to a fully-complementary target sequence of the guide sequence portion, wherein the guide sequence portion of the second RNA molecule differs from the guide sequence portion of the first RNA molecule.

16. (canceled)

17. (canceled)

18. (canceled)

19. (canceled)

20. A cell modified using the composition of claim 14.

21. (canceled)

22. (canceled)

23. The modified cell of claim 20, wherein the cell is a stem cell or any cell type capable of being reprogrammed to an induced pluripotent stem cell (iPSC).

24. (canceled)

25. (canceled)

26. (canceled)

27. Use of the composition of claim 14 for adoptive immunotherapy, comprising delivering the composition of claim 14, or a cell modified by the composition of claim 14, to a subject in need of adoptive immunotherapy.

28. (canceled)

29. (canceled)

30. (canceled)

31. (canceled)

32. (canceled)

33. A method of treating cancer, the method comprising delivering the composition of claim 14, or a cell modified by the composition of claim 14, to a subject in need of cancer treatment.

34. (canceled)

35. The method of claim 1, wherein the guide sequence portion of the RNA molecule further comprises 17-50 contiguous nucleotides containing nucleotides in the sequence set forth in any one of SEQ ID NOs: 658, 746, 2127, 2209, 4582, 5467, 6537, or 6831-6833.

36. The method of claim 35, wherein at least one CRISPR nuclease is OMNI-103.

37. The method of claim 10, wherein the guide sequence portion of the RNA molecule further comprises 17-50 contiguous nucleotides containing nucleotides in the sequence set forth in any one of SEQ ID NOs: 658, 746, 2127, 2209, 4582, 5467, 6537, or 6831-6833.

38. The method of claim 37, wherein at least one CRISPR nuclease is OMNI-103.

39. The composition of claim 14, wherein the guide sequence portion of the RNA molecule further comprises 17-50 contiguous nucleotides containing nucleotides in the sequence set forth in any one of SEQ ID NOs: 658, 746, 2127, 2209, 4582, 5467, 6537, or 6831-6833.

40. The composition of claim 39, wherein at least one CRISPR nuclease is OMNI-103.
